(19) United States
(12) Patent Application Publication
(10) Pub. No.: US 2019/0159925 A1
(43) Pub. Date: May 30, 2019

(54) URINE ABSORPTION PACK

(71) Applicant: Soon Keol KIM, Pyeongtaek-si (KR)

(72) Inventor: Soon Keol KIM, Pyeongtaek-si (KR)

(21) Appl. No.: 16/185,722

(22) Filed: Nov. 9, 2018

(30) Foreign Application Priority Data
Nov. 24, 2017 (KR) 20-2017-0006046

(51) Int. Cl. A61F 5/44; A61F 5/453; A61F 5/455; A61F 5/443

(52) U.S. Cl. CPC A61F 5/4401; A61F 5/453; A61F 5/443; A61F 5/4408; A61F 5/4407; A61F 5/4553

(57) ABSTRACT

The present invention relates generally to an urine absorption pack, and more specifically to a urine absorption pack that can be directly attached to (in the case of a man) or inserted into (in the case of a woman) the terminal urinary organ of a person who has difficulty urinating normally or whose urination occurs regardless of his or her intention, such as an infant, an old or weak person with mobility difficulties, a patient with dementia or urinary incontinence, or the like, and that can effectively absorb urine during urination.

### CROSS-REFERENCE TO RELATED APPLICATION

This application claims the benefit of Korean Patent Application No. 20-2017-0006046 filed on Nov. 24, 2017, which is hereby incorporated by reference herein in its entirety.

### BACKGROUND

### 1. Technical Field

The present invention relates generally to an urine absorption pack, and more specifically to a urine absorption pack that can be directly attached to (in the case of a man) or inserted into (in the case of a woman) the terminal urinary organ of a person who has difficulty urinating normally or whose urination occurs regardless of his or her intention, such as an infant, an old or weak person with mobility difficulties, a patient with dementia or urinary incontinence, or the like, and that can effectively absorb urine during urination.

### 2. Description of the Related Art

Generally, a person who has difficulty urinating normally or whose urination occurs regardless of his or her intention, such as an infant, an old or weak person with mobility difficulties, a patient with dementia or urinary incontinence, or the like, wears a diaper and deals with urine. However, a disadvantage arises in that that the diaper needs to be changed from time to time, and a problem arises in that as the amount of urine increases, the discomfort of the wearer becomes more serious.

In order to overcome the above problems, Korean Patent Application Publication No. 10-2009-0020440 (published on Feb. 26, 2009) proposes a sanitary convenience pack that is a urine collection container that can be used for a surgical patient or a patient with dementia or urinary incontinence. A user deals with urine via the inlet of the sanitary convenience pack, and an adhesive is attached to the inlet to temporarily store contents after use.

However, since the sanitary convenience pack proposed in Korean Patent Application Publication No. 10-2009-0020440 functions merely as a storage container for simply storing urine, a problem arises in that, when a user moves in the state in which urine in a liquid state has been stored in the convenient sanitary pack after urination, the urine in the liquid state is slopping in the sanitary convenience pack in conjunction with the movement, and thus the behavior of the user becomes inconvenient.

### SUMMARY

Accordingly, the present invention has been conceived to overcome the problems of the prior art, and an object of the present invention is to provide a urine absorption pack that can be mounted on the terminal urinary organ of a person who has difficulty urinating normally or whose urination occurs regardless of his or her intention, such as an infant, an old or weak person with mobility difficulties, a patient with dementia or urinary incontinence, or the like, and that can effectively absorb urine during urination.

In order to accomplish the above object, the present invention provides a urine absorption pack, including: an urine bag configured to be mounted on a penis, and provided with an inlet configured to receive urine discharged from the penis; an absorption member provided on the inside surfaces of the urine bag, and configured to absorb urine entering into the urine bag; and a fastening member provided on the inlet, and configured to fasten a penis inserted into the inlet by pressing the penis.

Preferably, the urine absorption pack further includes: a fastening element provided on the inlet, and configured to be inserted into a vagina; and a urine receiver provided on the fastening element, and configured to come into close contact with a urethral opening, from which urine is discharged, after the fastening element has been inserted into the vagina and to enable urine discharged from the urethral opening to flow into the urine bag.

Preferably, the fastening element is installed to be detachable from the inlet, and is separated from or inserted into the inlet in order to enable the penis to be inserted into the inlet when the urine absorption pack is mounted on the penis.

Preferably, the urine absorption pack further includes: a discharge hose configured to communicate with the urine bag and to discharge urine that has not been absorbed into the absorption member and remains in the urine bag; and a lid configured to close the outlet of the discharge hose.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view illustrating a urine absorption pack according to a first embodiment of the present invention;

FIG. 2 is a view showing a state in which the absorption member shown in FIG. 1 has been separated from the urine absorption pack;

FIG. 3 is a sectional view showing the sectional surface of the urine absorption pack shown in FIG. 1;

FIG. 4 is a view illustrating a urine absorption pack according to a second embodiment of the present invention;

FIG. 5 is an enlarged view illustrating the structure of a typical vulva;

FIG. 6 is a view illustrating a urine absorption pack according to the present invention of a third embodiment;

FIG. 7 is a view showing a state in which the fastening element shown in FIG. 6 has been inserted into an inlet; and

FIG. 8 is a view illustrating a urine absorption pack according to a fourth embodiment of the present invention.

### DETAILED DESCRIPTION

The advantages and features of the present invention and a method for achieving them will become apparent from the embodiments that will be described in detail below in conjunction with the accompanying drawings. However, the present invention is not limited to the embodiments disclosed below, but may be implemented in various different forms.

Throughout the present specification, embodiments are provided to make disclosure thorough and complete and to fully convey the scope of the invention to those having ordinary knowledge in the art to which the present invention pertains. Furthermore, the present invention is only defined by the scope of the claims. Accordingly, in some embodiments, well-known components, well-known operations, and well-known techniques will not be specifically described to prevent the present invention from being ambiguously interpreted.

Throughout the present specification, the same reference symbols refer to the same components. Furthermore, the terms used (described) herein are intended to illustrate embodiments, but are not intended to limit the present invention. In this specification, a singular form includes a plural form unless the context clearly dictates otherwise. Also, the terms “include,” “comprise” and their derivatives do not exclude the presence or addition of one or more other components and acts.

Unless otherwise defined, all the terms (including technical and scientific terms) used herein have the same meanings as being understood by those having ordinary knowledge in the technical field to which the present invention pertains. The terms should not be interpreted as having excessively ideal or formative meanings, unless definitely defined therein.

The technical features of the present invention will be described in greater detail with reference to the accompanying drawings.

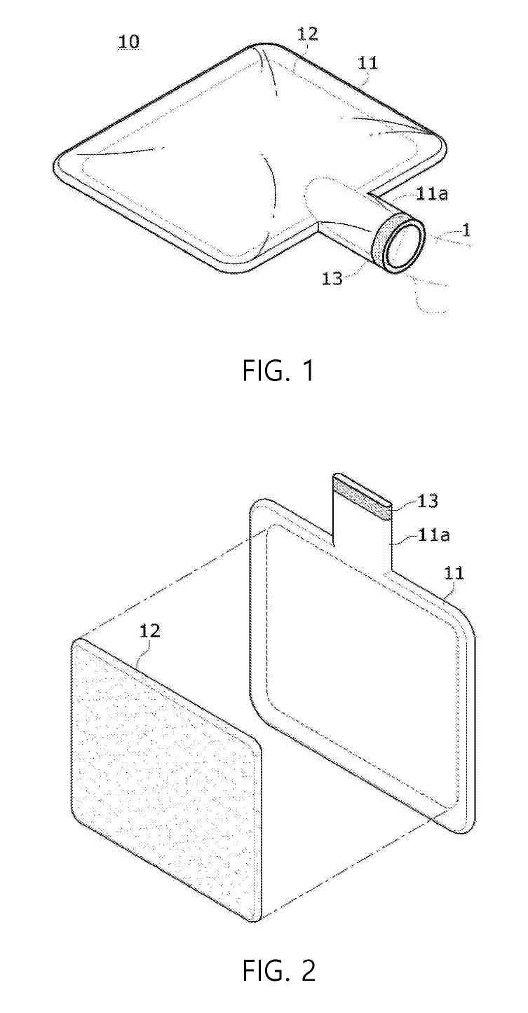

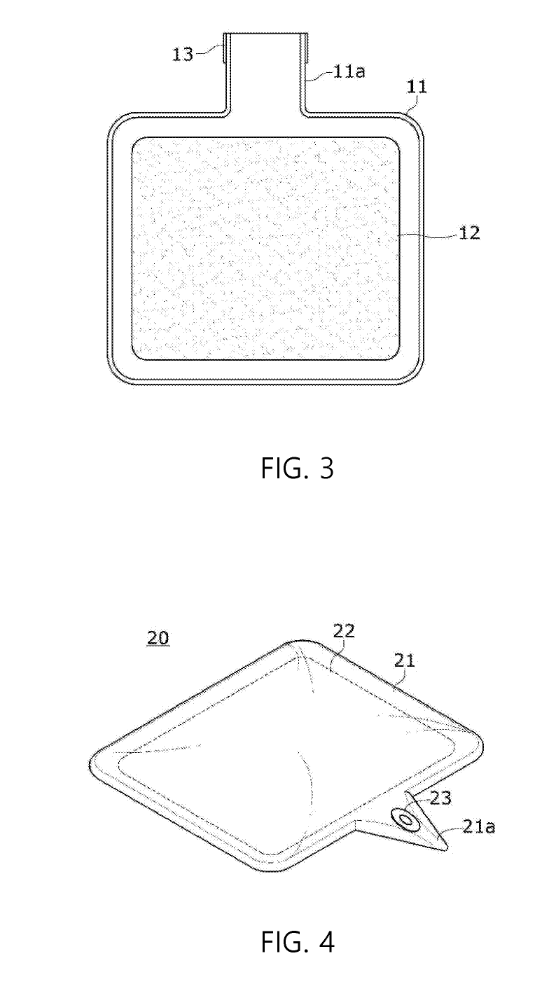

FIG. 1 is a view illustrating a urine absorption pack 10 according to a first embodiment of the present invention, FIG. 2 is a view showing a state in which the absorption member shown in FIG. 1 has been separated from the urine absorption pack 10, and FIG. 3 is a sectional view showing the sectional surface of the urine absorption pack 10 shown in FIG. 1.

Referring to FIGS. 1 to 3, the urine absorption pack 10 according to the first embodiment of the present invention is a man-only urine absorption pack, and includes a urine bag 11 configured to include an inlet 11a through which a penis 1 is inserted thereinto and to store urine discharged from the penis 1, an absorption member 12 provided on the inside surfaces of the urine bag 11 and configured to absorb urine discharged from the penis 1 into the urine bag 11, and a fastening member 13 provided on the inlet 11a of the urine bag and configured to fasten the penis 1 inserted into the inlet 11a by pressing the penis 1.

The urine bag 11 is made of synthetic resin material, preferably lightweight vinyl material, and includes the inlet 11a provided on at least one side thereof and configured to receive the penis 1. In this case, the inlet 11a may be made of the same vinyl material as the urine bag 11, or may be made of silicon material that is harmless to a human body when the penis 1 comes into direct contact with the inlet 11a.

Furthermore, the urine bag 11 may be provided with adhesive tape in order to attach the urine bag 11 to a human body, e.g., a thigh or groin portion, or may be provided with Velcro tape or a strap in order to tie and fasten the urine bag 11 to the thigh, waist or the like of a user.

As shown in FIGS. 2 and 3, the absorption member 12 is fastened to the inside surfaces of the urine bag 11 through attachment or stitching, and absorbs urine discharged into the urine bag 11. The absorption member 12 may be made of the same material as an absorption member that is used in a diaper or a sanitary pad.

As shown in FIGS. 1 and 2, the fastening member 13 is used to fasten the penis 1 in the state in which the penis 1 has been inserted into the inlet 11a of the urine bag 11 in order to prevent the urine bag 11 from being separated from the penis 1, and may be composed of an elastic band, Velcro tape, a strap, a belt, or the like.

As described above, the urine absorption pack 10 according to the first embodiment of the present invention is configured such that the absorption member 12 is provided inside the urine bag 11, and thus absorbs urine, discharged into the urine bag 11, via the absorption member 12 rather than simply storing urine discharged into the urine bag 11, thereby minimizing inconvenience attributable to the slopping of urine during the movement of a user.

Furthermore, the urine absorption pack 10 according to the first embodiment of the present invention is configured such that the fastening member 13 is provided on the inlet 11a of the urine bag 11, and fastens the penis 1 in the state in which the penis 1 has been inserted into the inlet 11a of the urine bag 11 in order to prevent the penis 1 from being easily separated from the urine bag 11, thereby preventing the urine bag 11 from being separated from the penis 1 even when a user wears the urine bag 11 for a long time.

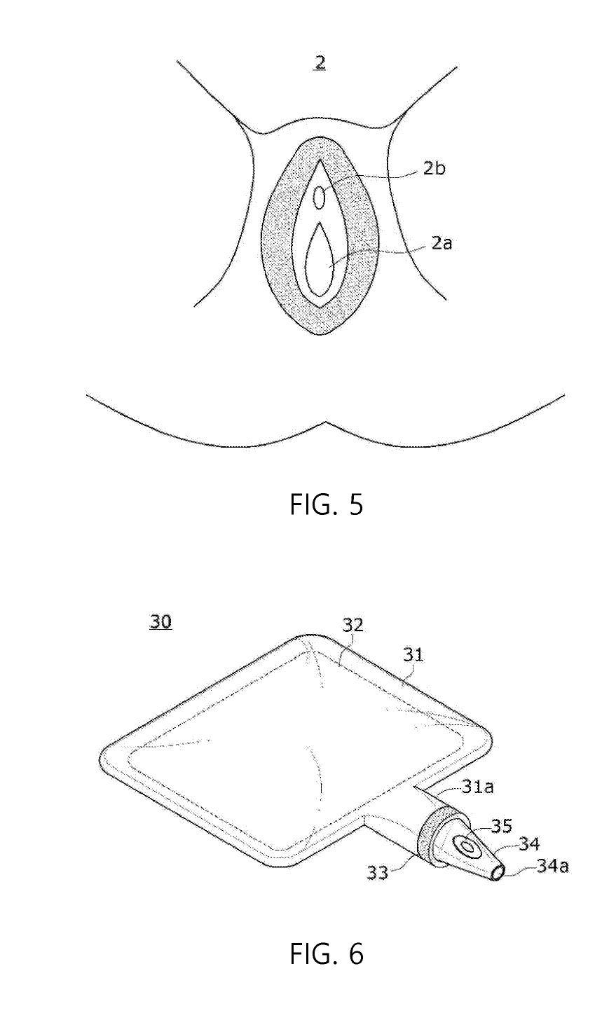

FIG. 4 is a view illustrating a urine absorption pack according to a second embodiment of the present invention, and FIG. 5 is an enlarged view illustrating the structure of a typical vulva.

Referring to FIGS. 4 and 5, the urine absorption pack 20 according to the second embodiment of the present invention is dedicated to a woman and mounted and used in a woman's vulva 2, and includes a urine bag 21 and an absorption member 22 provided inside the urine bag 21 in the same manner as the urine absorption pack 10 according to the first embodiment of the present invention.

Furthermore, the urine absorption pack 20 according to the second embodiment of the present invention is configured such that a fastening member 21a is provided on one side of the urine bag 21 and inserted and fastened into a woman's vagina 2a and such that a urine receiver 23 configured to come into close contact with the woman's urethral opening 2b and to receive urine discharged from the urethral opening 2b after the fastening element 21a configured to discharge urine has been inserted into the woman's vagina 2a is provided in the upper portion of the fastening element 21a.

The fastening element 21a may be made of vinyl material along with the urine bag 21, and may be made of silicon material harmless to a human body. Preferably, the fastening element 21a may be made of silicon material having predetermined rigidity so that the fastening element 21a can be inserted into the woman's vagina 2a, and is formed in a structure the width of which decreases toward the external end thereof so that the fastening element 21a can be stably inserted into the woman's vagina 2a.

The urine receiver 23 may be made of vinyl material having a thickness larger than that of the material of the urine bag 21, or may be made of silicon material harmless to a human body. Furthermore, urine discharged into the urine receiver 23 flows into the urine bag 21.

As shown in FIG. 4, the urine receiver 23 may be detachably attached to the outside surface of the fastening element 21a. The urine receiver 23 is preferably formed in a structure in which the width of the upper portion thereof is larger than that of the lower portion thereof, i.e., in an approximately funnel shape, in order to prevent urine discharged from the urethral opening 2b from flowing to the outside.

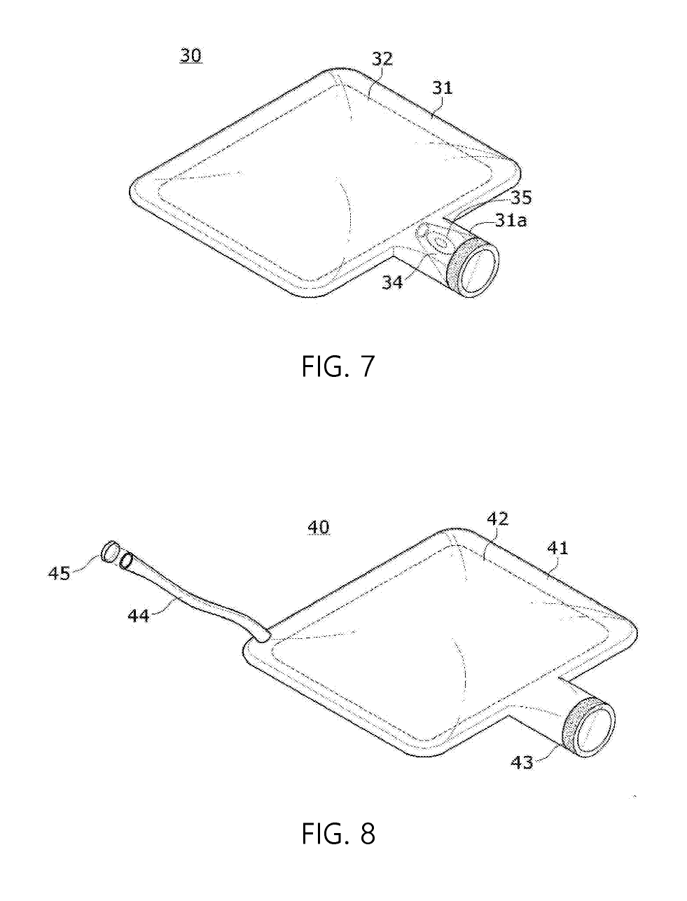

FIG. 6 is a view illustrating a urine absorption pack 30 according to the present invention of a third embodiment, and FIG. 7 is a view showing a state in which the fastening element shown in FIG. 6 has been inserted into an inlet.

Referring to FIGS. 6 and 7, the urine absorption pack 30 according to the third embodiment of the present invention has a structure formed by combining the man-only urine absorption pack 10 shown in FIG. 1 and the woman-only urine absorption pack 20 shown in FIG. 4 for unisex use. The urine absorption pack 30 includes a urine bag 31, an absorption member 32, and a fastening member 33, and further includes a fastening element 34 provided in an inlet 31a and configured to be inserted into a woman's vagina, and a urine receiver 35 provided on the fastening element 34. In this case, the urine bag 31, the absorption member 32, and the fastening member 33 have the same structures as those of the first embodiment.

The fastening element 34 is detachably installed in the inlet 31a of the urine bag 31. When the urine absorption pack 30 is used for the penis 1, the fastening element 34 may be separated from the inlet 31a or may be inserted into the inlet 31a, as shown in FIG. 7, so that the penis 1 can be inserted into the inlet 31a.

An inflow hole 34a is preferably formed in the external end of the fastening element 34 so that urine entering via the inlet 31a can flow into the urine bag 31 when the fastening element 34 is inserted into the inlet 31a of the urine bag 31, as shown in FIG. 7. It will be apparent that when the fastening element 34 is detachably attached to the inlet 31a, it is not necessary to additionally provide the inflow hole 34a.

A method of using the urine absorption pack 30 according to the embodiment of the present invention, which is configured as described above, is now described. When the urine absorption pack 30 is mounted and used in a woman's vulva, the urine absorption pack 30 is used in such a manner that the fastening element 34 is inserted and fastened into the woman's vagina 2a in a state in which the fastening element 34 has been projected from the inlet 31a of the urine bag 31, as shown in FIG. 6, and the urine receiver 35 is brought into close contact with and made to cover the woman's urethral opening 2b. In contrast, when the urine absorption pack 30 is mounted and used on the penis 1, the urine absorption pack 30 is used in such a manner that the fastening element 34 is inserted into the inlet 31a, as shown in FIG. 7, and the penis 1 is inserted into the inlet 31a.

FIG. 8 is a view illustrating a urine absorption pack 40 according to a fourth embodiment of the present invention.

Referring to FIG. 8, the urine absorption pack 40 according to the fourth embodiment of the present invention includes a urine bag 41, an absorption member 42, and a fastening member 43 in the same manner as in the first embodiment, and further includes a discharge hose 44 configured to communicate with the urine bag 41 and to discharge urine that has not been absorbed into the absorption member 42 and remains in the urine bag 41, and a lid 45 configured to close the outlet of the discharge hose 44.

When the amount of urine entering into the urine bag 41 is large and urine has not been completely absorbed into the absorption member 42, urine that has not been absorbed and remains in the urine bag 41 in a liquid state, and may cause inconvenience during the movement of a user. In order to prevent this inconvenience, the urine absorption pack 40 according to the fourth embodiment of the present invention further includes the discharge hose 44 configured to discharge part of urine, which has not been absorbed and remains, to the outside, and the lid 45 configured to close the discharge hose 44.

The discharge hose 44 and the lid 45 according to the fourth embodiment may be applied to the second embodiment and the third embodiment without change. Furthermore, the lid 45 is connected to the discharge hose 44 in order to prevent the lid 45 from being lost.

Meanwhile, although not shown in detail, each of the urine absorption packs 10, 20, 30 and 40 according to the first to fourth embodiments may further include a notice mark configured to indicate that urine has been discharged. In this case, the notice mark may be provided on the outside surface of each of the urine bags 11, 21, 31 and 41, and may be configured to detect the temperature of the urine bag 11, 21, 31 or 41 and to represent various colors.

As described above, according to the present invention, the following effects can be achieved.

First, according to the present invention, there is provided the urine absorption pack including the absorption member provided on the overall inside surfaces of the urine bag, so that the urine absorption pack absorbs urine, discharged into the urine bag, via the absorption member rather than simply storing urine discharged into the urine bag and thus urine discharged into the urine bag is absorbed into the absorption member, thereby minimizing inconvenience attributable to the slopping of urine during the movement of a user.

Second, according to the present invention, there is provided the urine absorption pack including the fastening member provided on the inlet of the urine bag, so that the fastening member fastens the urine bag in order to prevent the urine bag from being separated from a penis in the state in which the penis has been inserted into the inlet of the urine bag, thereby preventing the urine bag from being separated even when the urine bag is mounted on the penis and used for a long time.

Third, according to the present invention, there is provided the urine absorption pack further including the fastening element provided on the inlet and the urine receiver provided on the fastening element, so that the urine absorption pack can conveniently mounted and used for both a man and a woman as a single product, thereby improving usability.

Fourth, according to the present invention, there is provided the urine absorption pack further including the discharge hose configured to communicate with the urine bag and to discharge urine that has not been absorbed into the absorption member and remains in the urine bag and the lid configured to close the outlet of the discharge hose, so that there can be minimized urine that has not been completely absorbed into the absorption member and remains inside the urine bag in a liquid state because the amount of urine is large.

While the technical spirit of the present invention has been specifically described through the preferred embodiments, the preferred embodiments are intended to illustrate the technical spirit of the present invention, but are not intended to limit the technical spirit of the present invention. Accordingly, it should be understood by those having ordinary knowledge in the art to which the present invention pertains that various embodiments may be made by combining the embodiments of present invention within the scope of the technical spirit of the present invention.

What is claimed is:

1. A urine absorption pack, comprising:
an urine bag configured to be mounted on a penis, and provided with an inlet configured to receive urine discharged from the penis and at least any one of adhesive tape, Velcro tape, or a strap configured to fasten the urine absorption pack to a human body;
an absorption member provided on inside surfaces of the urine bag, and configured to absorb urine entering into the urine bag;
a fastening member provided on the inlet, and configured to fasten a penis inserted into the inlet by pressing the penis;
a fastening element provided on the inlet, configured to be inserted into a vagina, made of rigid silicon material, and formed in a structure a width of which decreases toward an external end; and
a urine receiver provided on the fastening element, and configured to come into close contact with a urethral opening, from which urine is discharged, after the fastening element has been inserted into the vagina and to enable urine discharged from the urethral opening to flow into the urine bag;
wherein the fastening element is installed to be detachable from the inlet, and is separated from or inserted into the inlet in order to enable the penis to be inserted into the inlet when the urine absorption pack is mounted on the penis, and an inflow hole is formed in an external end of the fastening element so that urine can flow into the urine bag when the fastening element is inserted into the inlet.

2. The urine absorption pack of claim 1, further comprising:
a discharge hose configured to communicate with the urine bag and to discharge urine that has not been absorbed into the absorption member and remains in the urine bag; and
a lid configured to close an outlet of the discharge hose.
